(19) United States
(12) Patent Application Publication
(10) Pub. No.: US 2022/0184214 A1
(43) Pub. Date: Jun. 16, 2022

(54) MULTICOMPONENT THERMOPLASTIC PRODUCT

(71) Applicant: PK MED, PARIS (FR)

(72) Inventors: FRÉDÉRIQUE GUILLAMOT, GERZAT (FR); MADIHA ALOUI DALIBEY, CLERMONT-FERRAND (FR)

(21) Appl. No.: 17/598,344

(22) PCT Filed: Mar. 27, 2020
(86) PCT No.: PCT/EP2020/058827

§ 371 Date: Sep. 27, 2021

(30) Foreign Application Priority Data
Mar. 28, 2019 (EP) 19305397.2

(51) Int. Cl. A61K 47/34; C08L 67/04; A61K 9/70; D01F 1/10; D01F 8/14

(52) U.S. Cl. CPC A61K 47/34; C08L 67/04; A61K 9/70; C08L 2203/02; D01F 1/10; D01F 8/14; A61K 9/7007

(57) ABSTRACT

The present invention relates to a new multicomponent plastic product comprising at least two different thermoplastic materials, wherein a first thermoplastic material comprises a first thermoplastic polymer, and a second thermoplastic material comprises a second thermoplastic polymer and a drug. According to the invention, the second thermoplastic material has a transformation temperature strictly lower than the transformation temperature of the first thermoplastic material and wherein the first and second plastic materials are at least partially adjacent in the multicomponent product.

The present invention relates to multicomponent thermoplastic products. More particularly, the present invention relates to multicomponent products comprising at least two distinct polymeric regions, wherein one of these polymeric regions contains a drug. The present invention also relates to a process for manufacturing multicomponent plastic products.

### BACKGROUND

Drug delivery compositions are well known in the medical field. Among them, drug delivery devices have been developed that allow to release, with more or less control rate, a drug in vivo. Most often, the drug is associated to a polymer, used as a vehicle for the drug.

Hot melt extrusion has been improved to be used for dispersing a drug into a polymer structure. The hot melt extrusion allows to prepare many dosage forms and formulations, such as granules, pellets, tablets, ophthalmic inserts, implants, stents or transdermal systems. This process shows several advantages compared to solvent-based production process, wherein solvents have to be removed by costly and time-consuming steps. However, hot melt extrusion involves heat treatment that may impact the activity of the drug.

Recently, a novel process for producing drug delivery compositions has been described in WO2019/020678. This process provides drug delivery compositions, wherein the drug is homogeneously dispersed and still exhibits some activity in the polymeric matrix. The drug delivery composition can thus be shaped in any drug delivery device with improved control release.

### SUMMARY OF THE INVENTION

The inventors now propose multicomponent plastic products that comprise at least two distinct plastic materials, composed of different thermoplastic polymers. A first plastic material is free of drug, whereas a second plastic material comprises a drug. More particularly, the drug is in a thermoplastic material with a transformation temperature below the transformation temperature of the thermoplastic material free of drug. According to the invention, these plastic materials are at least partially adjacent and are not intimately mixed. Indeed, the inventors have surprisingly discovered that the drug is able to spread from a region to an adjacent region. The inventors have also developed a process for manufacturing such multicomponent plastic products.

In this regard, it is an object of the invention to provide a multicomponent product comprising at least two different thermoplastic materials, wherein
a first thermoplastic material comprises a first thermoplastic polymer, and
a second thermoplastic material comprises a second thermoplastic polymer and a drug,
   wherein the second thermoplastic material has a transformation temperature (Tf) strictly lower than the transformation temperature (Tf) of the first thermoplastic material and wherein the first and second plastic materials are at least partially adjacent in the multicomponent product.

In a particular embodiment, the first thermoplastic polymer is PLA and the second thermoplastic polymer is PCL.

It is a further object of the invention to provide a process for manufacturing a multicomponent product according to the invention, comprising the steps consisting on

a. selecting a first thermoplastic material comprising a first thermoplastic polymer;

b. selecting a drug;

c. mixing the drug with a second thermoplastic polymer at a temperature at which said second thermoplastic polymer is in a partially or totally molten state, to obtain a second thermoplastic material, wherein the second thermoplastic material has a transformation temperature (Tf) strictly lower than the transformation temperature (Tf) of the first thermoplastic material;

d. coextruding or coinjecting or extruding coating said thermoplastic materials to obtain a multicomponent product.

These and the other objects and embodiments of the invention will become more apparent after the detailed description of the invention, including preferred embodiments thereof given in general terms.

### DETAILED INVENTION

### Definitions

The present disclosure will be best understood by reference to the following definitions.

A “thermoplastic material” refers to a composition under solid or molten state, comprising one or more thermoplastic polymer and optionally additional substances or additives, such as plasticizers, mineral or organic fillers, before any shaping or conditioning step. Preferably, a thermoplastic material comprises a mix of semi-crystalline and/or amorphous polymers, or semi-crystalline polymers. According to the invention, thermoplastic material is used for manufacturing plastic products.

Within the context of the invention, the term “multicomponent plastic product” or “multicomponent thermoplastic product” refers to any item or product (such as plastic sheet, film, tube, rod, profile, shape, massive block, fiber, filament, yarn, etc.) comprising at least two different thermoplastic materials, arranged relative to each other in such a way that they are not intimately mixed. For instance, some regions of the multicomponent plastic product may adhere to another or be bonded naturally due to plastic material compatibility or by use of additives between the two regions. Particularly, the multicomponent plastic product is a manufactured product, and more particularly a drug delivery device, such as implants, films, stents, leaflets, valves, coils, scaffolds, dressings, rods, patches, fibers, suture fibers, screws, bone plates, implants and prostheses.

A “polymer” refers to a chemical compound or mixture of compounds whose structure is constituted of multiple repeating units linked by covalent chemical bonds. A “thermoplastic polymer” refers to a polymer that becomes moldable above a specific temperature and solidifies upon cooling. Most often, a thermoplastic polymer is at least moldable at its melting temperature (Tm) and/or above. Some thermoplastic polymers begin to be moldable at their glass temperature (Tg). Within the context of the invention, the term thermoplastic polymer includes synthetic thermoplastic polymers, constituted of a single type of repeat unit (i.e., homopolymers) or of a mixture of different repeat units (i.e., copolymers). Synthetic thermoplastic polymers include thermoplastic polymers derived from petroleum oil or from bio-based materials, such as polyolefins, aliphatic or aromatic polyesters, polyamides, polyurethanes and vinyl polymers.

According to the invention, the term “transformation temperature” (Tf) corresponds to the temperature at which a thermoplastic material is in a partially or totally molten state, i.e. the temperature at which a thermoplastic material is fluid enough to be processed by extrusion or any heat treatment. A “transformation temperature” is also called “processing temperature” and is easily determined by the skilled artisan, taking account of the process of manufacturing and/or the components of the thermoplastic material, notably the Tf of the thermoplastic polymers contained in said thermoplastic material (said Tf being provided in technical datasheet of the thermoplastic polymers). As an example, the transformation temperature of thermoplastic material comprising mainly semi-crystalline polymers is generally a temperature close to or above the melting temperature (Tm) of said polymers, preferably above the Tm of said polymers. Regarding thermoplastic material comprising mainly amorphous polymers, the transformation temperature refers to the temperature at which the thermoplastic material is fluid enough to be processed by extrusion or any heat treatment (i.e., in a rubbery or softened state), i.e. also called softening temperature. Such temperature is generally higher than the glass transition temperature (Tg) of such amorphous polymer. When a thermoplastic material comprises more than one thermoplastic polymer, with different Tm (or softening temperature), the transformation temperature of the thermoplastic material is a temperature close to or above the Tm (or softening temperature) of the majority thermoplastic polymer. Alternatively, the transformation temperature of the thermoplastic material is a temperature close to or above the higher Tm (or higher softening temperature).

In particular embodiments, the transformation temperature of a thermoplastic material can be modulated (i.e., increased or decreased as compared to the Tf of the thermoplastic polymers of a thermoplastic material per se) by addition of additive(s) able to impact the polymer's fluidity.

As used herein, the term “drug” refers to a compound which has a physiological effect when administered to a subject. According to the invention, enzymes having a polymer degrading activity are not encompassed by the term “drug”.

As used herein, the terms “lower” or “below” a given temperature should be understood as “strictly lower” and “strictly below” respectively, i.e., said given temperature is not included.

As used herein, the term “by weight” means “based on the total weight” of the considered composition or product.

In the context of the invention, the term “about” refers to a margin of +/−5%, preferably of +/−1%, or within the tolerance of a suitable measuring device or instrument.

Plastic Materials

The present invention proposes to use at least two distinct thermoplastic materials, each comprising a thermoplastic polymer, to produce a plastic product that comprises at least two distinct parts, i.e. a multicomponent product. The distinct parts or regions are not melted within the plastic product but are maintained physically separated even if directly adjacent.

According to the invention, each plastic material comprises a thermoplastic polymer and one of them further comprises a drug. The thermoplastic material comprising the drug (herein after “the second thermoplastic material”) has a transformation temperature strictly below the transformation temperature of the other thermoplastic material (herein after “the first thermoplastic material”).

In a particular embodiment, the second thermoplastic material has a transformation temperature at least 50° C. below a transformation temperature of the first thermoplastic material, preferably 60° C., 70° C., 80° C. below.

In a particular embodiment, both thermoplastic materials comprise at least 70%, 75%, 80%, 85%, 90%, preferably at least 95% by weight of semi-crystalline thermoplastic polymers based on the total weight of polymers within the thermoplastic materials, and said semi-crystalline thermoplastic polymer of the second material has a melting temperature strictly below the melting temperature of said semi-crystalline thermoplastic polymer within the first thermoplastic material. Preferably, the semi-crystalline thermoplastic polymer of the second thermoplastic material has a Tm at least 50° C. below the Tm of the semi-crystalline thermoplastic polymer of the first thermoplastic material, more preferably 60° C., 70° C., 80° C. below.

It is therefore an object of the present invention to provide a multicomponent plastic product comprising at least two different thermoplastic materials, wherein
a first thermoplastic material comprises at least 70%, 75%, 80%, 85%, 90%, preferably at least 95% by weight of a first semi-crystalline thermoplastic polymer, based on the total weight of polymers within the first thermoplastic material, and
a second thermoplastic material comprises at least 70%, 75%, 80%, 85%, 90%, preferably at least 95% by weight of a second semi-crystalline thermoplastic polymer, based on the total weight of polymers within the second thermoplastic material, a drug,
   wherein the second semi-crystalline thermoplastic polymer has a melting temperature (Tm) below the melting temperature (Tm) of the first semi-crystalline thermoplastic polymer, and wherein the first and second thermoplastic plastic materials are at least partially adjacent in the multicomponent product.

In a particular embodiment, the second semi-crystalline thermoplastic polymer has a Tm at least 50° C. below the Tm of the first semi-crystalline thermoplastic polymer, preferably 60° C., 70° C., 80° C. below.

In a particular embodiment, both thermoplastic materials comprise a mix of amorphous and semi-crystalline polymers, and the polymer of the second material has a transformation temperature strictly below the transformation temperature of the polymer within the first thermoplastic material.

In a preferred embodiment, the thermoplastic polymers used for the two plastic materials are different from each other. For instance, the polymer of the first thermoplastic material is PLA and the polymer of the second thermoplastic material is PCL.

In another particular embodiment, the polymers of the thermoplastic materials are selected from polymers of same type (i.e., with same chemical formula) and with different grades, having different transformation temperatures. As well known by the skilled artisan, the grade of a polymer is defined according to the polymer's molecular mass, isomer rate, ramification, etc.). The final plastic product will thus be made of a single type of polymers, exhibiting different grades. For instance, the thermoplastic polymers are both PLA, wherein the first thermoplastic material comprises 4043D PLA grade from NatureWorks and the second thermoplastic material comprises PLA Luminy LX930 from Total Corbion.

In another particular embodiment, the polymers of the thermoplastic plastic materials are selected from polymers of same type and same grade but the second thermoplastic material further comprises components (e.g., plasticizers, fillers, or additional polymers) able to decrease the viscosity of said polymer, leading to a decrease in Tf of the second thermoplastic material as compared to the Tf of the first plastic material. For instance, the thermoplastic polymers are both selected from the same grade of PLA such as 4043D PLA grade from NatureWorks, but the second thermoplastic material further comprises a plasticizer so that its viscosity is decreased as compared to the viscosity of the first thermoplastic material.

In another embodiment, additives may be added to one or both thermoplastic materials in order to decrease the viscosities difference between both thermoplastic materials. Such adjustment may be obtained by addition of plasticizers, fillers, polymer(s) with higher or lower viscosity or any means known by the person skilled in the art to modify the viscosity of a polymer. Such addition may be performed during the production of each thermoplastic materials or directly during the step of production of the multicomponent plastic product.

First Thermoplastic Material

According to the invention, the first plastic material comprises a first thermoplastic polymer, and optionally additives or fillers. Preferably, the first plastic material comprises at least 70%, 75%, 80%, 85%, 90%, 95%, 99% by weight of a first thermoplastic polymer based on the total weight of the first plastic material. In a particular embodiment, the first plastic material comprises 100% wt of a first thermoplastic polymer. In another embodiment, the first plastic material comprises two or more thermoplastic polymers, and the first thermoplastic polymer (targeted by the degrading enzyme within the second plastic material) represents 70%, 75%, 80%, 85%, 90%, 95% by weight of the first thermoplastic material.

Advantageously, the first thermoplastic material has a transformation temperature (Tf) above 120° C., preferably above 150° C., more preferably above 180° C. Particularly, the first thermoplastic material comprises at least 70%, 75%, 80%, 85%, 90%, preferably at least 95% by weight of semi-crystalline thermoplastic polymer, based on the total weight of polymers in the first thermoplastic material, said semi-crystalline thermoplastic polymer having a melting temperature (Tm) above 120° C., preferably above 150° C., more preferably above 180° C.

Preferably, the first plastic material comprises at least 70%, 75%, 80%, 85%, 90%, 95%, 99% by weight of a first thermoplastic polymer, which is selected from semi-crystalline polymers having a Tm above 120° C., preferably above 150° C., more preferably above 180° C.

Preferably, the first thermoplastic polymer is selected from aliphatic, aromatic and/or aliphatic/aromatic polyanhydrides, such as (PCPP-SA)=poly(1,3 bis(p-carboxyphenoxypropane-sebacic acid) copolymer), polyolefins, aliphatic and semi-aromatic polyesters, polyorthoester polymers, polyamides, polyurethanes, vinyl polymers, polyethers or ester-ether copolymers and derivatives thereof. More preferably the first thermoplastic polymer is selected from aliphatic, aromatic and/or aliphatic/aromatic polyanhydrides, such as (PCPP-SA)=poly(1,3 bis(p-carboxyphenoxypropane-sebacic acid) copolymer), polyorthoester polymers, aliphatic and semi-aromatic polyesters, polyethers or ester-ether copolymers.

More preferably, the polyester is selected from, polylactic acid (PLA), poly(L-lactic acid) (PLLA), poly(D-lactic acid) (PDLA), poly(D,L-lactic acid) (PDLLA), stereocomplex PLA (scPLA), polyglycolic acid (PGA), polyhydroxy alkanoate (PHA), Poly(3-hydroxybutyrate) (P(3HB)/PHB), Poly(3-hydroxyvalerate) (P(3HV)/PHV), Poly(3-hydroxyhexanoate) (P(3HHx)), Poly(3-hydroxyoctanoate) (P(3HO)), Poly(3-hydroxydecanoate) (P(3HD)), Poly(3-hydroxybutyrate-co-3-hydroxyvalerate) (P(3HB-co-3HV)/PHBV), Poly(3-hydroxybutyrate-co-3-hydroxyhexanoate) (P(3HB-co-3HHx)/(PHBHHx)), Poly(3-hydroxybutyrate-co-5-hydroxyvalerate) (PHB5HV), Poly(3-hydroxybutyrate-co-3-hydroxypropionate) (PHB3HP), Polyhydroxybutyrate-co-hydroxyoctonoate (PHBO), polyhydroxybutyrate-co-hydroxyoctadecanoate (PHBOd), Poly(3-hydroxybutyrate-co-3-hydroxyvalerate-co-4-hydroxybutyrate) (P(3HB-co-3HV-co-4HB)), polybutylene succinate (PBS), polybutylen succinate adipate (PBSA), polybutylen adipate terephthalate (PBAT), polycaprolactone (PCL), poly(ethylene adipate) (PEA) and copolymers thereof such as poly(lactic-co-glycolic acid) copolymers (PLGA) or blends/mixtures of these materials. The polyethers may be selected e.g., from polyethylene glycol (PEG), preferably PEG with molecular mass above 600 g/mol, polyethylene oxide (PEO), or copolymers and blends/mixtures thereof. The ester-ether copolymers may be selected e.g., from polydioxanone (PDS).

In a particular embodiment, the first thermoplastic polymer is selected from copolymers of PLA and PGA (PLGA or PLA-co-PGA).

In a particular embodiment, the first thermoplastic polymer is selected from thermoplastic polymers having a transformation temperature (Tf) above 120° C., preferably above 150° C., more preferably above 180° C. and/or a glass transition temperature (Tg) above 10° C. In another particular embodiment, the first thermoplastic polymer is selected from semi-crystalline polymers having a melting temperature (Tm) above 120° C., preferably above 150° C., more preferably above 180° C. and/or a glass transition temperature (Tg) above 10° C. In another embodiment, the first thermoplastic polymer is selected from amorphous polymers having a softening temperature above 120° C. preferably above 150° C., more preferably above 180° C. and/or a glass temperature (Tg) above 10° C.

In a particular embodiment, the first thermoplastic polymer is selected from aliphatic polyester, preferably from polylactic acid (PLA).

The first plastic material may further comprise one or more additives. Generally speaking, the additives are used in order to enhance specific properties in the region of the plastic product made of the first plastic material. For instance, the additives may be selected from the group consisting without limitation of plasticizers, coloring agents, processing aids, rheological agents, anti-static agents, anti-UV agents, toughening agents, anti-fogging agents, compatibilizers, slip agents, flame retardant agents, anti-oxidants, light stabilizers, oxygen scavengers, inks, adhesives, fertilizers, and phytosanitary products. Advantageously, the first plastic material comprises less than 30% by weight (wt %) of such additives, preferably less than 15 wt %, more preferably less than 5 wt % or less than 1 wt %.

In a particular embodiment, a compatibilizer can be added to the first plastic material to increase adhesion between the first and second plastic materials. For instance, compatibilizers for polyesters may be selected from polyacrylates, ethylene terpolymers, acrylic esters, glycidyl methacrylate, triblock copolymers and/or maleic anhydride grafted polymer.

In a particular embodiment, the first plastic material comprises an anti-acid filler. The term “anti-acid filler” is used to designate a filler that has the ability to chemically neutralize an acid molecule, even contained in a plastic material (or produced by the degradation of such plastic material). The neutralizing reaction performed by an anti-acid filler is generally based on ion exchange. The presence of an anti-acid filler in a plastic material may help to increase and/or maintain the pH of the composition. Anti-acid fillers may be mineral or organic, synthetic or natural, and used alone or as a mix of several anti-acid fillers. The anti-acid filler can be selected from hydrotalcite, calcium carbonate, talc, mica, clay and/or calcium hydroxide. Preferably, the anti-acid filler represents between 0.1% and 30% by weight of the first plastic material, based on the total weight of the plastic material, preferably between 2 wt % and 15 wt %, more preferably between 1 wt % and 10 wt %.

Advantageously, the first thermoplastic material comprises less than 30 wt % of both additives and anti-acid fillers, preferably less than 15 wt %, more preferably less than 5 wt % or less than 1 wt %.

Second Thermoplastic Material

According to the invention, the second plastic material comprises a second thermoplastic polymer, a drug and optionally additives and/or fillers. Preferably, the second plastic material comprises at least 50%, 60%, 70% by weight of a second thermoplastic polymer, based on the total weight of the thermoplastic material. The additives and/or fillers can be same as or different from the additives and fillers of the first plastic material, including compatibilizers. Advantageously, the second plastic material comprises less than 30% by weight of such additives and/or fillers, based on the total weight of the thermoplastic material preferably less than 15 wt %, more preferably less than 5 wt % or less than 1 wt %.

Advantageously, the second thermoplastic material has a transformation temperature (Tf) below 180° C., preferably a Tf below 150° C., more preferably a Tf below 120° C., even more preferably below 100° C. Particularly, the second thermoplastic material comprises at least 70%, 75%, 80%, 85%, 90%, preferably at least 95% by weight of semi-crystalline thermoplastic polymers, based on the total weight of polymers in the second thermoplastic material, said semi-crystalline thermoplastic polymer having a melting temperature (Tm) below 180° C., preferably below 150° C., more preferably below 120° C.

Preferably, the second plastic material comprises at least 70%, 75%, 80%, 85%, 90%, 95%, 99% by weight of a second thermoplastic polymer, which is selected from semi-crystalline polymers having a Tm below 180° C., preferably below 150° C., more preferably below 120° C.

According to the invention, the second plastic material comprises at least one thermoplastic polymer, different from the thermoplastic polymer of the first plastic material (e.g., chemically different, different for grades, and/or differently formulated to reach different viscosities).

Advantageously, the second thermoplastic polymer is selected from thermoplastic polymers having a transformation temperature (Tf) below 180° C. and/or a glass transition temperature (Tg) below 70° C. Preferably, the second thermoplastic polymer is selected from thermoplastic polymers having a Tf below 150° C. and/or a Tg below 30° C. More preferably, the second thermoplastic polymer is selected from thermoplastic polymers having a Tf below 120° C. In another particular embodiment, the second thermoplastic polymer is selected from semi-crystalline polymers having a melting temperature (Tm) below 180° C., preferably below C150° C., more preferably below 120° C. and/or a Tg below 70° C., preferably below 30° C. In another embodiment, the second thermoplastic material comprises a mix of semi-crystalline and amorphous second thermoplastic polymer, wherein the mix of polymers has a Tf below 180° C., preferably below 150° C., more preferably below 120° C. and/or a Tg below 70° C., preferably below 30° C. In another embodiment, the second thermoplastic polymer is selected from amorphous polymers having a softening temperature below 180° C., preferably below 150° C., more preferably below 120° C. and/or a Tg below 70° C., preferably below 30° C.

In a particular embodiment, the second thermoplastic polymer is a polyester and/or polyether, preferably selected from polycaprolactone (PCL), poly butylene succinate adipate (PBSA), polybutylene adipate terephthalate (PBAT), polydioxanone (PDS), polyhdroxyalkanoate (PHA), polylactic acid (PLA), polyglycolic acid (PGA), polyethylene glycol (PEG), preferably PEG with molecular mass above 600 g/mol, polyethylene oxide (PEO) or copolymers thereof. In a preferred embodiment, the second polymer is a polyester, preferably selected from polycaprolactone (PCL), poly butylene succinate adipate (PBSA), polybutylene adipate terephthalate (PBAT), polyhdroxyalkanoate (PHA), polylactic acid (PLA), polyglycolic acid (PGA), or copolymers thereof. In a particular embodiment, the first polymer is a copolymer of polylactic acid and polyglycolic acid (PLGA or PLA-co-PGA). In another particular embodiment, the second polymer is PCL.

In another embodiment, the second thermoplastic polymer is selected from aliphatic, aromatic and/or aliphatic/aromatic polyanhydrides, such as (PCPP-SA)=poly(1,3 bis(p-carboxyphenoxypropane-sebacic acid) copolymer), and polyorthoester polymers.

The second plastic material preferably contains at least 0.001% by weight of drug, based on the total weight of the second plastic material. In a particular embodiment, the second plastic material comprises between 0.001 to 50 wt %, preferably between 0.01 wt % and 30 wt % of drug, more preferably between 0.1 wt % and 30% wt, even more preferably between 0.5 wt % and 20 wt %, or between 0.5 and 10 wt %. In a particular embodiment, the second plastic material comprises about 1% by weight of drug.

Preferably, the drug is selected from pharmaceutical compounds, peptides, proteins, antibiotics, analgesics, vaccines, vaccine adjuvants, anti-inflammatory agents, anti-tumor agents, hormones, cytokines, anti-fungal agents, anti-viral agents, anti-bacterial agents, anti-diabetics, steroids, specific enzyme inhibitor, growth stimulating agent, immunosuppressors, immuno-modulators, anti-hypertensive drugs, anti-arrhythmic drugs, inotropic drugs, addiction therapy drugs, anti-epileptic drugs, anti-aging drugs, drugs to treat neuropathies or pain, hypolipemic drugs, anti-coagulants, antibodies or antibody fragments, antigens, anti-depressant or psychotropic agents, neuro-modulators, drugs for treating a disease selected from brain disease, liver disease, pulmonary disease, cardiac disease, gastric disease, intestine disease, ovary disease, testis disease, urological disease, genital disease, bone disease, muscle disease, endometrial disease, pancreatic disease and/or renal disease, ophthalmic drugs, anti-allergic agents, contraceptive or luteinizing agents and mixtures of at least two of these drugs, with the provision that said drug does not exhibit any polymer degrading activity.

In a particular embodiment, the drug is chosen among compounds having therapeutic or prophylactic purposes in a mammal, and more particularly in a human.

Advantageously, the drug has a molecular mass between 5 kDa and 200 kDa. In a particular embodiment, the drug is a protein having a molecular mass between 50 kDa and 150 kDa, between 75 kDa and 150 KDa, between 100 KDa and 150 Kda, between 120 KDa and 150 KDa, between 50 KDA and 100 KDa, between 50 KDa and 75 KDa. In another embodiment, a protein having a molecular mass between 30 kDa and 50 kDa. In another embodiment, a hormone having a molecular mass between 5 kDa and 30 kDa, between 10 KDa and 30 KDa, between 15 KDa and 30 KDa, between 20 KDa and 30 KDa.

The denaturation temperature corresponds to the temperature at which half of the drug loses activity. Preferably, the drug is chosen among compounds with a denaturation temperature equal to or above 50° C. In a particular embodiment, the drug is chosen among compounds having a denaturation temperature between 50° C. and 140° C., for instance about 120° C.

In a particular embodiment, the second plastic material further comprises a degrading enzyme selected among enzymes able to degrade the first thermoplastic polymer. Examples of suitable degrading enzymes include, without limitation, depolymerase, hydrolase, esterase, lipase, cutinase, protease, polyesterase, carboxylesterase, oxygenase and/or oxidase such as laccase, peroxidase or oxygenase. The enzymes may be in pure or enriched form (such as concentrated supernatant), or mixed with other excipients or diluents. A combination of enzymes may be used as well. The person skilled in the art will be able to select the appropriate degrading enzyme depending on the nature of the thermoplastic polymer of the first plastic material that must be degraded.

For instance, when the first thermoplastic polymer is PLA, the degrading enzyme may be selected from a protease, preferably selected from Amycolatopsis sp., Amycolatopsis orientalis, proteinase K from Tritirachium album, Actinomadura keratinilytica, Laceyella sacchari LP175, Thermus sp., Bacillus licheniformis, Bacillus thermoproteolyticus or any commercial enzymes known for degrading PLA such as Savinase®, Esperase®, Alcalase®, Everlase®, Protex®, Optimase®, Multifect® or any enzymes from the family of the subtilisin CAS 9014-01-1 or any functional variant thereof. Examples of suitable proteases are described in WO 2016/062695, WO 2016/146540 or WO 2018/109183). If required, the commercial enzymes may be reformulated (e.g., diafiltrated to remove commercial carrier) before to be mixed with the second thermoplastic polymer. Examples of such reformulations are described in WO 2019/043145.

In a particular embodiment, said degrading enzyme is further able to degrade the polymer of the second thermoplastic material.

In another embodiment, the second thermoplastic material comprises a degrading enzyme selected from enzymes able to degrade the first thermoplastic polymer and an additional degrading enzyme selected from enzymes able to degrade the second thermoplastic polymer. For instance, the second thermoplastic material comprises PCL, an enzyme able to degrade PCL (e.g., lipase) and a protease able to degrade the PLA of the first thermoplastic material.

In another embodiment, a compatibilizer can be added to the second plastic material to increase the adhesion with the second plastic material. For instance, compatibilizers for polyesters may be selected from polyacrylates, ethylene terpolymers, acrylic esters, glycidyl methacrylate, triblock copolymers, maleic anhydride grafted polymer.

In a particular embodiment, the second plastic material comprises an anti-acid filler. Preferably, the anti-acid filler represents between 0.1% and 30% by weight of the second plastic material, based on the total weight of the plastic material, preferably between 2 wt % and 15 wt %.

In a particular embodiment, the second plastic material comprises, based on the total weight of the second plastic material:
(i) from 50 to 99.9 wt % of at least one polyester, preferably PCL, PLA or PLGA;
(ii) from 0 to 29.9 wt % of at least one anti-acid filler and/or additives including compatibilizers, preferably selected from hydrotalcite, calcium carbonate, talc, mica, clay and/or calcium hydroxide;
(iii) from 0.1 to 50 wt % of drug.

Multicomponent Plastic Products

It is the purpose of the present invention to provide new multicomponent plastic products that comprise two or more thermoplastic materials, which are composed of different thermoplastic polymers and wherein one of the thermoplastic materials includes a drug.

According to the invention, the multicomponent product comprises at least two different thermoplastic materials, wherein a first thermoplastic material comprises a first thermoplastic polymer and a second thermoplastic material comprises at least a second thermoplastic polymer and at least one drug and wherein the second thermoplastic material has a transformation temperature (Tf) strictly lower than the transformation temperature (Tf) of the first thermoplastic material and wherein the first and second thermoplastic materials are at least partially adjacent in the multicomponent product.

According to the invention, the at least two thermoplastic materials are arranged within the multicomponent plastic product in different parts or regions physically distinct from each other, i.e. the parts or regions are not intimately mixed. In the context of the invention, a region refers to a part of the plastic product comprising a given thermoplastic polymer, that may be different from a thermoplastic polymer of at least one adjacent region of said plastic product. The physical contact between two adjacent regions allows a drug to spread from one region to an adjacent one.

In a particular embodiment, the plastic product is a bicomponent plastic product made with two distinct thermoplastic materials, as exposed above. Said two distinct thermoplastic materials may be arranged in two or more distinct parts. According to the invention, at least two parts made with distinct plastic materials are adjacent. Advantageously, the multicomponent product is produced by coextrusion or coinjection or extrusion coating.

Advantageously, the multicomponent product is selected from multicomponent filaments and multilayer plastic products.

In an embodiment, the multicomponent product is a multicomponent filament. Advantageously, the multicomponent filament is produced by coextrusion.

For instance, the multicomponent product is a bicomponent filament composed of a first and a second thermoplastic materials, wherein the resulting cross-section of the bicomponent filament may resemble a variety of different configurations. For example, the first region and the second region of the bicomponent filament may be arranged in the form of a sheath/core, tipped tri-lobal, bicomponent tri-lobal, side-by-side, islands in the sea, or a segmented pie. In a particular embodiment, the first and a second thermoplastic materials are arranged in the form of sheath/core. In another particular embodiment, the thermoplastic materials are arranged in the form of segmented pie or island(s) in the sea, to improve the drug accessibility to the first polymer.

In a particular embodiment, the multicomponent product is a bicomponent filament that comprises a sheath made of the first plastic material and a core made of the second plastic material. Alternatively, the bicomponent filament comprises a core made of the first plastic material and a sheath made of the second plastic material.

In a particular embodiment, the multicomponent product is a multicomponent filament with diameter below 5 mm, preferably below 2 mm. Such multicomponent filament may be used for the production of subsequent plastic articles using 3D printing or any means suitable for shaping a plastic article (e.g., a drug delivery device with particular shape). Examples of cross-sections of such multicomponent filaments include, without limitation, sheath/core, side-by-side, islands in the sea, or segmented pie cross-sections.

In another embodiment, the multicomponent product is a multicomponent filament with a diameter below 250 μm, preferably below 50 μm. Advantageously, such kind of filaments present circular (sheath/core, side-by-side, islands in the sea, or segmented pie) or tri-lobal cross-sections. Such multicomponent filament may be used for manufacturing yarns. The yarns are produced using meltspinning extruder to extrude together many dozens of filaments, leading to the production of multifilament yarns, each filament being a multicomponent plastic product. In a particular embodiment, the yarns can be used for wound dressing, or the like. Non-woven articles may also be produced directly by spunlaid (spunbond or meltblown) of filaments.

In another embodiment, the multicomponent product is selected from multilayer plastic products, including multilayer plastic films, multilayer plastic sheets, multilayer profiles and tubes.

In a particular embodiment, the multicomponent product is a multilayer plastic product, wherein at least one layer made of second plastic material is adjacent to at least one layer made of first plastic material. The multicomponent product may comprise additional layers or regions of any kind (plastic material, metal compound such as aluminum or foil, glass fiber or carbon fiber, etc.). The layers may have different lengths and/or thicknesses. The layers may overlap entirely or partially. The layers may be bonded with specific adhesive, during the manufacturing, through dedicated processes or may adhere to each other naturally due to polymer compatibility.

In another particular embodiment, the multilayer plastic product comprises at least one layer made of second plastic material sandwiched between two layers made of first plastic material.

Advantageously, the weight ratio first plastic material/second plastic material of the multicomponent product is comprised between 50/50 and 99/1, between 60/40 and 98/2 or between 70/30 and 95/5. In a particular embodiment, the multicomponent product is a bicomponent filament comprising a core of first plastic material and a sheath of second plastic material, wherein the core represents between 30 and 40% by weight of the total weight of the bicomponent filament and the sheath represents 60 to 70% by weight of the total weight of the bicomponent filament. In another particular embodiment, the multicomponent product is a bicomponent filament comprising a core of first plastic material and a sheath of second plastic material, wherein the core represents between 5 and 25% by weight of the total weight of the bicomponent filament and the sheath represents 75 to 95% by weight of the total weight of the bicomponent filament.

In a particular embodiment, the multicomponent product is a multilayer plastic product, wherein the at least one layer of second plastic material represents between 1 and 50%, preferably between 1 and 20% by weight of the total weight of the multilayer plastic product and the at least one layer of first plastic material represents between 50 to 99%, preferably between 80 and 99% by weight of the total weight of the bicomponent filament.

In a particular embodiment, the multicomponent product is a multilayer plastic product, wherein the at least one layer of second plastic material sandwiched between two layers of first plastic material represents between 1% and 50%, preferably between 1% and 20% by weight of the total weight of the multilayer plastic product and the two layers of first plastic material represents between 50% and 99%, preferably between 80% and 99% by weight of the total weight of the bicomponent filament.

In a particular embodiment, the multicomponent product comprises at least two thermoplastic materials, wherein the first thermoplastic polymer is PLA and the second thermoplastic polymer is PCL.

In a particular embodiment, the multicomponent product is a multilayer plastic product, wherein at least one layer made of second plastic material comprises PCL and a drug and at least one layer made of first plastic material comprises at least PLA. In a particular embodiment, at least one layer made of second plastic material comprising PCL and a drug is sandwiched between two layers made of first plastic material comprising PLA.

In a particular embodiment, the multicomponent product is a multicomponent filament, wherein the second plastic material comprises PCL and a drug and the first plastic material comprises PLA.

In a preferred embodiment, the multicomponent product is a drug delivery device. More particularly, the multicomponent plastic product is shaped as a drug delivery device. The drug delivery device may consist on one or more multicomponent filaments, one or more multilayers films, rods, massive blocks, etc., with a final design of implant, film, stent, leaflet, valve, coil, scaffold, dressing, rod, patch, fibers, suture fibers, screw, bone plate, implant, prosthesis, etc. It is thus also a purpose of the invention to provide a new drug delivery device allowing to release, preferably in a controlled rate, a drug that is embedded into the polymeric matrix (i.e., thermoplastic material) of said delivery device.

Process of Manufacturing

It is the purpose of the invention to provide a process for preparing a multicomponent plastic product having one or more first regions, made of a first plastic material, at least partially adjacent to one or more second regions, made of a second plastic material.

According to the invention, the process of manufacturing a multicomponent product comprises the steps consisting of

a. selecting a first plastic material comprising a first thermoplastic polymer;

b. selecting a drug;

c. mixing the drug with a second thermoplastic polymer at a temperature at which said second thermoplastic polymer is in a partially or totally molten state, to obtain a second plastic material wherein the second thermoplastic material has a transformation temperature (Tf) strictly lower than the transformation temperature (Tf) of the first thermoplastic material;

d. coextruding or coinjecting or extruding coating said plastic materials to obtain a multicomponent product.

Preferably the second thermoplastic material has a transformation temperature at least 50° C. below the transformation temperature of the first thermoplastic material, preferably 60° C., 70° C., 80° C. below.

In a particular embodiment, the first thermoplastic material comprises at least 70%, 75%, 80%, 85%, 90%, preferably at least 95% by weight of a first semi-crystalline thermoplastic polymer based on the total weight of polymers in the first thermoplastic material, and the second thermoplastic material comprise at least 70%, 75%, 80%, 85%, 90%, preferably at least 95% by weight of a second semi-crystalline thermoplastic polymer based on the total weight of polymers in the second thermoplastic material, and the second semi-crystalline thermoplastic polymer has a melting temperature below the melting temperature of the first semi-crystalline thermoplastic polymer.

The drug is mixed with the second thermoplastic polymer and optional additives, fillers and/or degrading enzyme at a temperature at which said second thermoplastic polymer is in a partially or totally molten state. Methods for introducing drugs within a plastic material are described for instance in WO 2019/020679 and WO 2019/020678-.

Advantageously, the drug is mixed with the second thermoplastic polymer in an amount between 0.001 to 50% by weight, based on the total weight of the second plastic material, preferably between 0.1% to 30% by weight of drug. In a particular embodiment, the second plastic material is prepared by introducing the drug in the second thermoplastic polymer during heating the second thermoplastic polymer. More generally speaking, the step of preparation of the second plastic material is performed at a temperature at which the second polymer is in a partially or totally molten state, so that the drug is embedded into the second polymer. Preferably, such preparation is performed by extrusion. Alternatively, such preparation is performed by internal mixing or co-kneading.

In a preferred embodiment, the second plastic material is prepared by introducing the second thermoplastic polymer and the drug in an extruder, such as a twin-screw extruder. The resulting extrudate may be granulated into solid pellets and optionally dried, until residual humidity decreases below 5%, preferably below 2%, more preferably below 1%, even more preferably below 0.5%.

Such pellets, or more generally the second plastic material, can then be used to prepare the multicomponent product.

According to the invention, the multicomponent plastic product is obtained by shaping the first and second plastic material during heat treatment. Advantageously, both the first and the second plastic materials are in partially and/or totally molten state.

In a particular embodiment, the heat treatment is a co-extrusion treatment, wherein the two plastic materials are introduced in separate extruders, melted and joined in a common die. Advantageously, each extruder comprises one or more heating zones, wherein temperature can be managed independently. It is thus possible to adapt the temperatures within each extruder to the thermoplastic polymer of the considered plastic material. Each plastic material is thus in partially or totally molten state when arriving within the common die.

In a particular embodiment, the temperature within the common die corresponds to the highest temperature between the two extruders, so that both plastic materials are maintained in partially and/or totally molten state. Accordingly, the second plastic material, which contains the drug, is submitted to the highest temperature only during the final step of shaping. In another particular embodiment, the temperature within the common die may be a temperature between the lowest and the highest temperatures of both extruders, provided that both plastic materials are maintained in partially and/or totally molten state in the die.

In a particular embodiment, the first thermoplastic polymer is PLA and the second thermoplastic polymer is PCL. The processing temperature (temperature within the extruders) for PLA is between 120° C. and 180° C., +/−10° C., whereas the processing temperature for PCL is between 60° C. and 110° C., +/−5° C., and the temperature in the common die is 180° C., +/−10° C.

In another particular embodiment, the first thermoplastic polymer is PLA and the second thermoplastic polymer is PCL. The processing temperature (temperature within the extruders) for PLA is between 190° C. and 230° C., +/−10° C., whereas the processing temperature for PCL is between 70° C. and 120° C., +/−5° C., and the temperature in the common die is 190° C., +/−10° C.

More generally speaking, the first and second plastic materials have preferably close viscosity when arriving in the common die. In the context of the invention, the viscosity of a thermoplastic polymer and of the plastic material containing said thermoplastic polymer is the quantity that describes a fluid's resistance to flow. Formally, viscosity is the ratio of the shearing stress to the velocity gradient in a fluid. It is thus possible to manufacture a plastic.

In a particular embodiment, the step of shaping the plastic product is implemented by the common die or spinning pack of the extruders in the coextrusion process. A person skilled in the art would adapt the die or the spinning pack of the co-extruder to the shape needed. The extruder can comprise a coextrusion die selected from sheet die, flat-film die, circular film die, pipe or tubing die, profile extrusion die. For instance, a vertical die with small diameter holes produces bicomponent or multicomponent multifilament textile, i.e. a textile product consisting of several bicomponent or multicomponent filaments. A horizontal die with one or several holes produces multicomponent textile filaments or 3D printing filament. A flat die with an elongated slot produces multilayer films or sheets for thermoforming applications. An annular die in the blow-molding extrusion produces multilayer films. Other dies with particular geometry produce tubes or multilayer profiles.

In a particular embodiment, the co-extruder comprises a spinning pack or die with multifilament or monofilament spinnerets, to produce bicomponent or multicomponent 3D printing monofilament, bicomponent or multicomponent monofilament textile or bicomponent or multicomponent multifilament textile, spunbond or meltblown nonwovens.

In another particular embodiment, the heat treatment is an extrusion coating process, wherein one plastic material is forced by an extruder to go through a horizontal die onto a moving solid sheet or film composed of the other plastic material.

In another particular embodiment, the heat treatment is a co-injection treatment, wherein the two plastic materials are introduced in separate extruders ending in two independent injection ports. Advantageously, each extruder comprises one or more heating zones, wherein temperature can be managed independently. It is thus possible to adapt the temperatures within each extruder to the thermoplastic polymer of the considered plastic material. Each plastic material is thus in partially or totally molten state when arriving at each injection port. The first plastic material and the second plastic material can be injected simultaneously or sequentially in the mold.

1-15. (canceled)

16. A multicomponent plastic product comprising at least two different thermoplastic materials, wherein
a first thermoplastic material comprises a first thermoplastic polymer, and
a second thermoplastic material comprises a second thermoplastic polymer and a drug, wherein the second thermoplastic material has a transformation temperature (Tf) lower than the transformation temperature (Tf) of the first thermoplastic material and wherein the first and second plastic materials are at least partially adjacent in the multicomponent product.

17. The multicomponent plastic product according to claim 16, wherein the second thermoplastic material has a transformation temperature (Tf) below 180° C.

18. The multicomponent plastic product according to claim 16, wherein the second thermoplastic polymer is selected from a polyester and/or polyether or is selected from polycaprolactone (PCL), polybutylene succinate adipate (PBSA), polybutylene adipate terephthalate (PBAT), polydioxanone (PDS), polyhydroxyalkanoate (PHA), polylactic acid (PLA), polyglycolic acid (PGA), polyethylene glycol (PEG), PEG with molecular mass above 600 g/mol, polyethylene oxide (PEO) or copolymers thereof.

19. The multicomponent plastic product according to claim 16, wherein the first thermoplastic polymer is selected from polyolefins, aliphatic and semi-aromatic polyesters, polyamides, polyurethanes, vinyl polymers and derivatives thereof.

20. The multicomponent plastic product according to claim 16, wherein the first thermoplastic polymer is selected from aliphatic and semi-aromatic polyesters and polyamides.

21. The multicomponent plastic product according to claim 16, wherein the first thermoplastic material/second thermoplastic material weight ratio is 50/50 to 99/1, 60/40 to 98/2 or 70/30 to 95/5.

22. The multicomponent plastic product according to claim 16, wherein said multicomponent product is selected from multicomponent filament or a multilayer plastic product.

23. The multicomponent plastic product according to claim 16, wherein said multicomponent product is a multicomponent filament, wherein the first and second thermoplastic materials are arranged in the form of a sheath/core, tipped tri-lobal fiber, bicomponent tri-lobal, side-by-side, islands in the sea or segmented pie.

24. The multicomponent plastic product according to claim 16, wherein said multicomponent product is a multicomponent filament wherein the first thermoplastic material is a sheath made with the first thermoplastic polymer and the second thermoplastic material is a core made of the second thermoplastic polymer and drug.

25. The multicomponent plastic product according to claim 16, wherein the first thermoplastic polymer is PLA and the second thermoplastic polymer is PCL.

26. The multicomponent plastic product according to claim 16, wherein the drug is selected from pharmaceutical compounds, peptides, proteins, antibiotics, analgesics, vaccines, vaccine adjuvants, anti-inflammatory agents, anti-tumor agents, hormones, cytokines, anti-fungal agents, anti-viral agents, anti-bacterial agents, anti-diabetics, steroids, specific enzyme inhibitor, growth stimulating agent, immunosuppressors, immuno-modulators, anti-hypertensive drugs, anti-arrhythmic drugs, inotropic drugs, addiction therapy drugs, anti-epileptic drugs, anti-aging drugs, drugs to treat neuropathies or pain, hypolipemic drugs, anti-coagulants, antibodies or antibody fragments, antigens, anti-depressant or psychotropic agents, neuro-modulators, drugs for treating a disease selected from brain disease, liver disease, pulmonary disease, cardiac disease, gastric disease, intestine disease, ovary disease, testis disease, urological disease, genital disease, bone disease, muscle disease, endometrial disease, pancreatic disease and/or renal disease, ophthalmic drugs, anti-allergic agents, contraceptive or luteinizing agents and mixtures of at least two of these drugs, with the provision that the drug does not exhibit any polymer degrading activity.

27. The multicomponent plastic product according to claim 16, wherein said multicomponent product is a medical device selected from implant, film, stent, leaflet, valve, coil, scaffold, dressing, rod, patch, fibers, suture fibers, screw, bone plate or implant, bone cement and prostheses.

28. A process for manufacturing a multicomponent product comprising:
a) selecting a first thermoplastic material comprising a first thermoplastic polymer;
b) selecting a drug;
c) mixing the drug with a second thermoplastic polymer at a temperature at which said second thermoplastic polymer is in a partially or totally molten state, to obtain a second thermoplastic material, wherein the second thermoplastic material has a transformation temperature (Tf) lower than the transformation temperature (Tf) of the first thermoplastic material; and
d) coextruding or coinjecting or extruding coating said thermoplastic materials to obtain a multicomponent product.

29. The process according to claim 28, wherein the step of coextruding is performed within a coextruder with a coextrusion die selected from sheet die, flat-film die, pipe or tubing die, profile extrusion die.

30. The process according to the claim 28, wherein the drug is mixed with the second thermoplastic polymer in an amount between 0.01 and 30% by weight, based on the total weight of the second thermoplastic material.
